(19) United States
(12) Patent Application Publication
(10) Pub. No.: US 2008/0154379 A1
(43) Pub. Date: Jun. 26, 2008

(54) Interbody fusion hybrid graft

(75) Inventors: Anton J. Steiner, Wharton, NJ (US); Gary Thomas, Ringwood, NJ (US); Dennis McBride, Cranford, NJ (US)

Correspondence Address: JONES DAY, 222 EAST 41ST ST, NEW YORK, NY, 10017, US

(73) Assignee: Musculoskeletal Transplant Foundation

(21) Appl. No.: 11/643,994

(22) Filed: Dec. 22, 2006

(51) Int. Cl. A61F 2/44

(52) U.S. Cl. 623/17.16

(57) ABSTRACT

The invention is directed toward a sterile composite bone graft for use in implants comprising a central member constructed of biocompatible plastic with two end caps of cortical bone mated to opposite ends of the central member. The central member is cylindrically ring shaped with a plurality of ribs formed in the side wall of the cylinder.

### RELATED APPLICATION

There are no related applications.

### STATEMENT REGARDING FEDERALLY SPONSORED RESEARCH OR DEVELOPMENT

Not applicable.

### REFERENCE TO SEQUENCE LISTING, A TABLE OR A COMPUTER PROGRAM LISTING COMPACT DISC APPENDIX

None.

### FIELD OF INVENTION

The present invention is generally directed toward a surgical implant and more specifically is a shaped composite bone block implant having a synthetic central portion and allograft cortical end caps for the fusion of vertebral bones when the implant is introduced between adjacent vertebrae to be fused.

### BACKGROUND OF THE INVENTION

The use of substitute bone tissue dates back around 1800. Since that time research efforts have been undertaken toward the use of materials which are close to bone in composition to facilitate integration of bone grafts. Developments have taken place in the use of grafts to use materials such as corals, hydroxyapatites, ceramics or synthetic materials such as biodegradable polymer materials. Surgical implants should be designed to be biocompatible in order to successfully perform their intended function. Biocompatibility may be defined as the characteristic of an implant acting in such a way as to allow its therapeutic function to be manifested without secondary adverse affects such as toxicity, foreign body reaction or cellular disruption.

Human allograft tissue is widely used in orthopaedic, neuro-, maxillofacial, podiatric and dental surgery. The tissue is valuable because it is biocompatible, strong, biointegrates in time with the recipient patient's tissue and can be shaped either by the surgeon to fit the specific surgical defect or shaped commercially in a manufacturing environment.

Allograft bone is a logical substitute for autologous bone. It is readily available and precludes the surgical complications and patient morbidity associated with obtaining autologous bone as noted above. Allograft bone is essentially a collagen fiber reinforced hydroxyapatite matrix containing active bone morphogenic proteins (BMP) and can be provided in a sterile form. The demineralized form of allograft bone is naturally both osteoinductive and osteoconductive. The demineralized allograft bone tissue is fully incorporated in the patient's tissue by a well established biological mechanism. It has been used for many years in bone surgery to fill the osseous defects previously discussed.

Allograft bone occurs in two basic forms; cancellous and cortical.

Many devices of varying shapes and forms have been fabricated from allograft cortical tissue by machining. Surgical implants such as pins, rods, screws, anchors, plates, intervertebral spacers and the like have been made and used successfully in human surgery. These pre-engineered shapes are used by the surgeon in surgery to restore defects in bone to the bone's original anatomical shape.

Injury or disease to the head, neck, or shoulders can cause abnormal forces to be applied on the cervical vertebra. This situation is often treated surgically by a procedure intended to fuse the two adjacent cervical or spinal vertebrae to each other. Such fusion relieves the pressure the partially displaced vertebrae place on the adjacent spinal nerves.

Many surgical devices have been developed and used successfully to immobilize and fuse the misaligned vertebrae. Metal plates screwed into the adjacent vertebrae work well, but after time post-operatively, the stress rise occurring at the screw position causes erosion of the bone and resultant slipping. This has been improved by placing load-bearing spacers between the two (or more) misaligned vertebrae. The spacer is both load-bearing and of a material which will induce, or at least support, fusion between the vertebrae.

Removal of damaged or diseased discs, restoration of disc space height and fusion of adjacent vertebrae to treat chronic back pain and other ailments are known medical techniques. Implants such as intervertebral spacers are often implanted in the disc space engaging the vertebrae to maintain or reestablish disc space height after removal of all or a portion of the disc. The spacers are formed of a variety of both resorbable and non-resorbable materials, including, for example, titanium, surgical steel, polymers, composites and bone. It is currently considered desirable to promote fusion between the vertebral bodies that are adjacent to the damaged or diseased discs. Typically, an osteogenic material is combined with a spacer and inserted in the disc space to facilitate and promote bone growth. While the selection of the implant configuration and composition can depend upon a variety of considerations, it is often desirable to select a resorbable material that does not shield the bone ingrowth. Bone and bone-derived components can provide suitable material to prepare the implants. However, bone material and in particular cortical bone acceptable for use in implants is a scarce resource, being derived from limited number human tissue donor resources.

Suitable bone or bone-derived material for use in implants, in general, is almost exclusively obtained from allograft and xenograft sources, both of which come from a limited supply. Since intervertebral spacers must withstand the compressive loads exerted by the spine, these implants are often cortical bone which has the mechanical strength suitable for use in any region of the spine. Cortical spacers are often shaped from cortical long bones, which are primarily found in the lower limbs and include, for example, femur, fibula, and the tibia bones. However, these long bones make up only a fraction of the available bone source. The scarcity of desired donor bone makes it difficult to provide implants having the desired size and configuration for implantation between vertebrae, which can require relatively large implants. It is further anticipated that as the population ages there will be an increased need for correction for spinal deformities and a concomitant increase in the demand for bone-derived components. Therefore, these structural bone portions must be conserved and used efficiently to provide implants. The scarcity of suitable bone material has also hindered efforts to design and manufacture varying configurations of suitable implants for arthodesis of the spine. Further, various implant configurations have not been physiologically possible to obtain given the structural and geometrical constraints of available donor bone.

One known treatment for fusing two vertebrae is the insertion of a suitably shaped dowel into a prepared cylindrical cavity which reaches the two vertebrae to be fused. The dowel used is preshaped allograft bone.

A number of allograft bone spacers have been used in surgery as spacers. They are commonly called the ACF spacer constructed as a cortical bone cross section, shaped like a washer with teeth to discourage graft explusion and an axial center hole; a VG3 cervical spacer constructed with two ramp shaped cortical plates held together with cortical pins, the top and bottom surfaces being ridged to discourage graft expulsion; an ICW spacer constructed with an elongated C spaced cortical portion with a cancellous inside to allow rapid ingrowth (slice of iliac crest) and a SBS spacer constructed with a single piece cortical member with serrated top and bottom surfaces and an axial center hole.

The ICW (iliac crest wedge) has been used for a long time for cervical spine fusion and has a total load bearing force around 4500 Newtons. Testing has noted that cervical vertebrae fail in compression at about 2000 Newtons. The ICW spacer suffers from high unit variability because of its natural, anatomic variations.

U.S. Pat. No. 5,972,368 issued on Oct. 26, 1999 discloses the use of cortical constructs (e.g. a cortical dowel for spinal fusion) which are cleaned to remove all of the cellular material, fat, free collagen and non-collagenous protein leaving structural or bound collagen which is associated with bone mineral to form the trabecular struts of bone. The shaped bone is processed to remove associated non-collagenous bone proteins while maintaining native bound collagen materials and naturally associated bone minerals. The surface of a machined cortical bone is characterized by a wide variety of openings resulting from exposure by the machining process of the Haversian canals present throughout cortical bone. These canals serve to transport fluids throughout the bone to facilitate the biochemical processes that occur at variable angles and depths within the bone.

An attempt to solve the increasing bone supply problems using a combined cortical and cancellous bone block is shown in U.S. Pat. No. 4,950,296 issued Aug. 21, 1990 which uses a cubically configured cortical shell defining a through going internal cavity and a cancellous plug fitted into the cavity so that the end surfaces of the cancellous plug are exposed. Another reference, WIPO Patent Publication Number WO 02/24122 A2, published Mar. 28, 2002 show various intervertebral spacers formed of cortical and cancellous bone composites such as sandwiches, with intersecting ribs and rods.

U.S. Pat. No. 6,294,187 issued Sep. 25, 2001 is directed toward a shaped osteimplant of compressed bone particles. The shaped implant is disc shaped and has a number of holes drilled therein for macroporosity and the holes can be filled with an osteogenic putty material.

Conversely, WIPO Patent Publication Number WO 02/07654 A2, published Jan. 31, 2002 discloses intervertebral spacers formed of dense cancellous human or animal bone. In one embodiment, a cortical rod or cortical rods are placed in bores cut through a cancellous bone block to provide load bearing strength with the ends of the rods being exposed on both sides of the cancellous bone block. Another embodiment shows a C shaped cortical block with a cancellous plug inserted into the recess of the C to form a rectangular spacer. A pin is inserted through a bore cut through the legs of the C block and through the cancellous plug to keep the cancellous plug positioned with the recess of the cortical component.

U.S. Pat. No. 6,379,385 issued Apr. 30, 2002 also discloses the use of a spongy block having a plurality of cortical rods mounted in through going bores cut through the bone block. In another embodiment, a X-shaped cortical support member is mounted therein to provide structured strength to the composite implant.

It is also known to mate various bone components together to form a single implant. In this regard, see, Albee, Bone Graft Surgery in Disease, Injury and Deformity, (1940), pp. 30, which uses a tongue nd groove and dove tail to hold separate pieces of bone together for implant use, and U.S. Publication No. US2002/0029084 A1, published Mar. 7, 2002, which shows a three component implant with a center core surrounded by two outer semicircular portions. The outer portions have alternative dove tail joints on adjacent bone portions to secure the outer portions together forming a dowel shaped bone implant.

In posterior lumbar interbody fusion (“PLIF”) two adjacent vertebral bodies are fused together by removing the affected disc and inserting an implant that would allow for bone to grow between the two vertebral bodies to bridge the gap left by the disc removal. Consequently, there is a need for an implant which should have a load bearing compressive strength but uses a minimal amount of allograft bone. More specifically, there is a need for an implant that is an integrated implant formed with two or more components that are interlocked to form a mechanically effective, strong unit.

### SUMMARY OF THE INVENTION

The composite allograft cervical fusion block is directed toward a three piece, mated bone fusion block or spacer constructed with a central member of load bearing plastic material with two ring shaped end cap members of cortical bone mounted to the central member for use in orthopedic surgical procedures. Each cap member defines a dove tail shaped projection extending from its planar proximal r surface with the plastic middle member having a dove tail recess cut in both end surfaces to receive the dove tail projection of the cortical cap member. The central member is cylindrical with a ring shaped cross section with the side wall being formed with opposing open support ribs.

It is an object of the invention to use a bone block geometry to provide a composite bone block of plastic and cortical bone components having performance characteristics that meet or exceed conventional spinal fusion requirements.

It is another object of the invention to utilize a shaped cortical plastic implant block which provides the mechanical strength characteristics that can withstand compression forces and provide overall strength and durability to the structure.

It is still another object of the invention to provide a spinal fusion implant which uses a load bearing plastic component member to take up the high forces which can arise between two vertebral bodies and cortical cap members to accelerate the healing process.

It is yet another object of the invention to provide a pre-machined shaped allograft bone structure which can effectively promote new bone growth and accelerate healing.

Currently available allografts are mechanically mated section of bone material, resulting in use of a limited supply of material and the allograft cannot be customized for specific patients spinal anatomy.

There is a need for new approaches to providing tissues, in particular allograft tissues as there is a need for an implant that allows more efficient use of source material. There is thus a need for an implant that is an integrated implant using minimal allograft bone that are interlocked to form a mechanically effective strong unit for fusing vertebrae.
These and other objects, advantages, and novel features of the present invention will become apparent when considered with the teachings contained in the detailed disclosure. This disclosure, along with the accompanying drawings and description, constitutes a part of this specification and illustrates embodiments of the invention which serve to explain the principles of the invention.

### BRIEF DESCRIPTION OF THE DRAWINGS

FIG. 1 is a perspective view of the inventive interbody fusion hybrid graft implant;

FIG. 2 is an exploded perspective view of the interbody fusion hybrid graft implant shown in FIG. 1;

FIG. 3 is a cross sectional view taken along lines 3′-3′ of FIG. 1 and

FIG. 4 is an exploded cross sectional view of the exploded perspective view of FIG. 2.

### DETAILED DESCRIPTION OF THE INVENTION

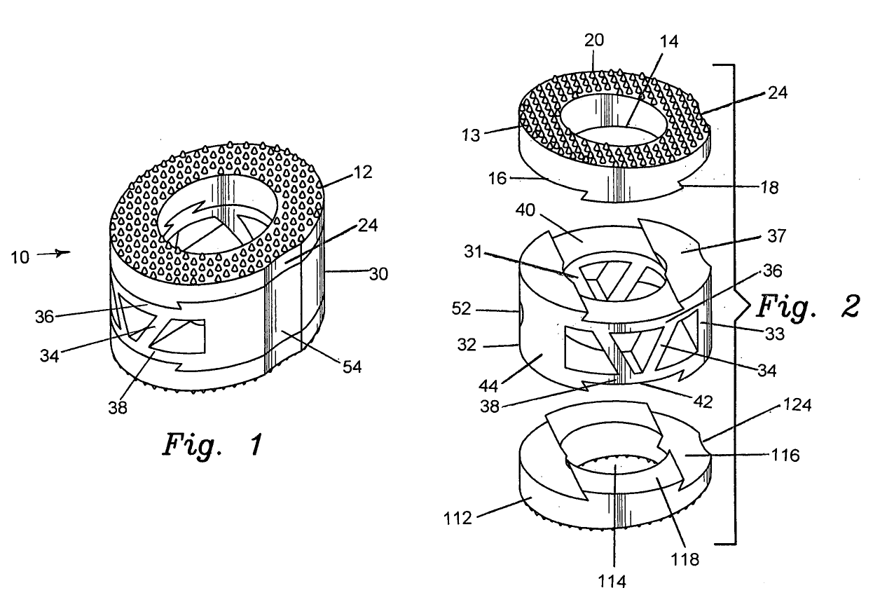

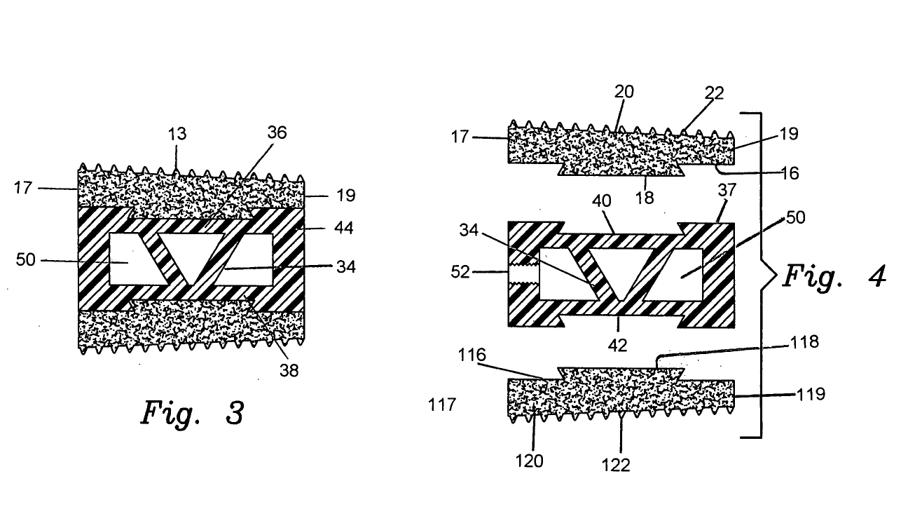

The preferred embodiment and best mode of the present invention is shown in FIGS. 1 through 4. The composite bone implant block 10 is shown in FIG. 1 in accordance with the present invention.

The composite cortical bone block body or intervertebral spacer 10 is preferably constructed with a first end cap member 12 constructed of cortical bone taken from donors cut into a ring shape. The cap member body 13 has an interior circular through going bore 14 formed or cut therein, and defines a flat planar bottom surface 16 which is provided with a dove tail shaped projection 18 which extends outward from the planar bottom surface 16. The cap body is tapered with the rear end 17 being of a greater height than the front end 19. The outer or top surface 20 which is tapered has a plurality of teeth 22 formed or cut into the exterior surface to provide a gripping surface on the adjacent vertebrae. The taper runs between 5° to 10° and the height of the upper cap member runs between 3-4 mm. The side wall of the ring body is formed with a channel or groove 24. The cortical cap members 20 and 120 have superior wall strength for support between load bearing body structures such as vertebrae. While it is noted that the bottom wall surfaces and are planar, these surfaces can be provided with any kind of complementary construction.

The middle or center support member 30 has a cylindrical ring shaped body 32 with cylindrical throughgoing bore 31 and is constructed of a biocompatible plastic such as poly ether ether ketone (PEEK), a crystalline polymer material which expands when it comes into contact with water or other fluids. The ring wall 32 has a plurality of wall V shaped ribs 34 formed in the side between the dove tail shaped recesses 40 and 42 which interconnect top planar section 36 and bottom planar section 38. The center support member 30 has a height ranging from 11 to 24 m. However, other polymeric molded material with similar mechanical properties can be used. The molded poly middle section is offered in a full range of heights and footprints (IE; ALIF, PLIF, TPLIF, ACF,) to cover the entire size range for the specific fusion procedures (cervical, thoracic or lumbar) anterior, posterior or other approach. Cut into the top surface 37 of the top planar section 36 and the bottom section 38 are respective dove tail shaped recesses 40 and 42 respectively. The ribs 34 are formed along the same longitudinal axis as the dove tail shaped recesses. The cylindrical side wall 44 together with the top planar section 36 and the bottom planar section 38 form a central cavity or chamber 50. A locking inserter bore 52 is cut into the side wall 44 transverse the axis of the dovetail recess to receive an inserter locking mechanism. A channel 54 is seen in FIG. 1 cut in the side wall and mates with channels 24 and 124 of the end caps.

The bottom cortical end cap member 112 of cortical bone is cut into a generally cylindrical ring shape with a tapered top surface and a dovetail extending from the bottom surface. The cap member body 113 has an interior circular throughgoing bore 114 cut therein, and defines a flat planar bottom surface 116 which is provided with a dove tail shaped projection 118 which extends outward from the bottom surface 116. The bottom surface 116 is tapered with the rear end 117 being of a greater height than the front end 119. The outer surface 120 which is tapered has a plurality of teeth 122 formed or cut into the exterior surface to provide a gripping surface on the adjacent vertebrae. The taper runs between 5° to 10° and the height of the second cap member runs between 3-4 mm.

The cortical ca[member 20 and 120 have superior wall strength for support between load bearing body structures such as vertebrae and has a compressive load together with the center member 30 in excess of 3000 Newtons. The composite implant body 10 height can range from 8-12 mm preferably 10 mm depending upon patient needs with a corresponding length ranging from 12 to 20 mm, preferably 16 mm with a width ranging from 10 mm to 14 mm preferably 12 mm, again depending upon surgeon preference and the size of the fusion block which will be used on the individual patient. The central member 30 expands when contacted with fluid thus firmly holding the implant between the two vertebrae and also tightly holds the end cap member 20 and 120 in the respective recesses. The dovetail projections may have been slightly reduced in size during the lypolization process.

While this application has been discussed in terms of using the preferred embodiment namely, allograft cortical cap members of the bone blocks, alternative sources of the components of the components of the bone blocks may be substituted such as xenograft bone or synthetic graft materials. With any of these alternatives, the bone blocks may be shaped as described above. The devices provide the surgeon with a graft that has the combined and best characteristics of allograft bone materials.

The cap member of the present invention were prepared by machining cortical bone taken from any acceptable donor. Suitable bones used for the cortical cap members are the radius, ulna, femur, tibia, humerus and the talus.

The unique features of allograft bone that make it desirable as a surgical material are, its ability to slowly resorb and be integrated into the space it occupies while allowing the bodies own healing mechanism to restore the repairing bone to its natural shape and function by a mechanism known in the art as creeping substitution.

It is well known that bone contains osteoinductive elements known as bone morphogenetic proteins (BMP). These BMP's are present within the compound structure of cortical bone and are present at a very low concentrations, e.g. 0.003%. BMP's direct the differentiation of pluripotential mesenchymal cells into osteoprogenitor cells which form osteoblasts. The ability of freeze dried demineralized bone to facilitate this bone induction principle using BMP present in the bone is well known in the art. However, the amount of BMP varies in the bone depending on the age of the bone donor and the bone processing. Based upon the work of Marshall Urist as shown in U.S. Pat. No. 4,294,753, issued Oct. 13, 1981 the proper demineralization of cortical bone will expose the BMP and present these osteoinductive factors to the surface of the demineralized material rendering it significantly more osteoinductive. The removal of the bone mineral leaves exposed portions of collagen fibers allowing the addition of BMP's and other desirable additives to be introduced to the demineralized outer treated surface of the bone structure and thereby enhances the healing rate of the cortical bone in surgical procedures.

It is also possible to add one or more rhBMP's to the bone by soaking and being able to use a significantly lower concentration of the rare and expensive recombinant human BMP to achieve the same acceleration of biointegration. The addition of other useful treatment agents such as vitamins, hormones, antibiotics, antiviral and other therapeutic agents could also be added to the bone or placed in a container or host material in the chamber 53 of the center member 30.

Any number of medically useful substances can also be incorporated in the chamber created in the center segment and the same could be filled with bone substitute, bioglass and with the addition of medically useful substances to the same. Such substances include collagen and insoluble collagen derivatives, hydroxyapatite and soluble solids and/or liquids dissolved therein. Also included are antiviricides such as those effective against HIV and hepatitis; antimicrobial and/or antibiotics such as erythromycin, bacitracin, neomycin, penicillin, polymyxin B, tetracycline, viomycin, chloromycetin and streptomycin, cefazolin, ampicillin, azactam, tobramycin, clindamycin, gentamycin and silver salts. It is also envisioned that amino acids, peptides, vitamins, co-factors for protein synthesis; hormones; endocrine tissue or tissue fragments; synthesizers; enzymes such as collagenase, peptidases, oxidases; polymer cellpl scaffolds with parenchymal cells; angiogenic drugs and polymeric carriers containing such drugs; collagen lattices; biocompatible surface active agents, antigenic agents; cytoskeletal agents; cartilage fragments, living cells and cell elements such red blood cells, white blood cells, platelets, blood plasma, pluripotential cells, chondrocytes, bone marrow cells, mesenchymal stem cells, osteoblasts, osteoclasts and fibroblasts, epithelial cells and endothelial cells present as a concentration of 10⁵ and 10⁶ per cc of a carrier, natural extracts, tissue transplants, bioadhesives, transforming growth factor (TGF-beta), insulin-like growth factor (IGF-1); platlet derived growth factor (PDGF), fibroblast growth factor (FGF) (numbers 1-23), osteopontin, vascular endothelial growth factor (VEGF), growth hormones such as somatotropin, cellular attractants and attachment agents, blood elements; natural extracts, tissue transplants, bioadhesives, bone digestors; antitumor agents; fibronectin; cellular attractants and attachment agents; immuno-suppressants; permeation enhancers, e.g. fatty acid esters such as laureate, myristate and stearate monoesters of polyethylene glycol, enamine derivatives, alpha-keto aldehydes can be added to the composition.

While the present invention is described for use in the cervical spine, it is also suitable for use in the lumbar and/or thoracic spine. Th implant can be provided in a variety of sizes, each size configured to be inserted between a specific pair of adjacent vertebrae. For example, the implant can be provided in selected dimensions to maintain disc height, correct lordosis, kyphosis or other spinal deformities.

The principles, preferred embodiments and modes of operation of the present invention have been described in the foregoing specification. However, the invention should not be construed as limited to the particular embodiments which have been described above. Instead, the embodiments described here should be regarded as illustrative rather than restrictive. Variations and changes may be made by others without departing from the scope of the present invention as defined by the following claims:

1. A sterile composite graft comprising:
a central biocompatible cylindrical ring shaped member with coupling means formed on opposite ends, allograft bone cap members mounted on either end of said central member to said coupling means, said central member defining a central throughgoing chamber with support ribs formed in a side wall of said central member.

2. A sterile composite graft as claimed in claim 1 wherein at least one cap member is constructed of cortical bone.

3. A sterile composite graft as claimed in claim 1 wherein each cap member defines a protruding dovetail shaped structure and said central member has a dovetail recess formed in each end surface which can receive and hold said dovetail shaped structure.

4. A sterile composite graft as claimed in claim 1 wherein both cap members are constructed of cortical bone and define a flat planar bottom surface with coupling means formed therein

5. A sterile composite graft as claimed in claim 1 wherein said central member is constructed of ceramic.

6. A sterile composite graft as claimed in claim 1 wherein said central member is constructed of biocompatible plastic.

7. A sterile composite graft as claimed in claim 6 wherein said biocompatible plastic is PEEK.

8. A sterile composite graft as claimed in claim 1 wherein said allograft cap member is ring shaped with a tapered thickness and has teeth on its outer surface.

9. A sterile composite graft as claimed in claim 1 wherein at least one of said graft members is provided with a cellular material additive taken from a group consisting of living cells and cell elements such red blood cells, white blood cells, platelets, blood plasma, pluripotential cells, chondrocytes, bone marrow cells, mesenchymal stem cells, osteoblasts, osteoclasts and fibroblasts, epithelial cells and endothelial cells present as a concentration of 10⁵ and 10⁶ per cc of a carrier.

10. A sterile composite graft as claimed claim 1 wherein at least one of said graft members has an additive taken from a group of growth factors consisting of transforming growth factor (TGF-beta), insulin-like growth factor (IGF-1); platlet derived growth factor (PDGF), fibroblast growth factor (FGF) (numbers 1-23), osteopontin, vascular endothelial growth factor (VEGF), growth hormones such as somatotropin cellular attractants and attachment agents.

11. A sterile composite graft as claimed claim 1 wherein at least one of said graft members has an additive taken from a group of additives consisting of antimicrobials effective against HIV and hepatitis and antimicrobial and/or antibiotics consisting of erythromycin, bacitracin, neomycin, penicillin, polymyxin B, tetracycline, viomycin, chloromycetin and streptomycin, cefazolin, ampicillin, azactam, tobramycin, clindamycin, gentamycin and silver salts.

12. A sterile composite graft as claimed in claim 1 wherein said central member defines a bore through a side wall transverse to a central axis of said central member.

13. A sterile composite bone graft for use in implants comprising:
a load bearing ring shaped center member constructed of biocompatible plastic defining opposing end planar surfaces with a dovetail mounting recess formed in each of said planar surfaces, said ring shaped center member defining a plurality of ribs in a side wall, a plurality of cap members mounted to said ring shaped center member, each of said cap members being constructed of allograft bone and inclined to form a tapered height, each said cap member defining a flat proximal surface with a dovetail shaped projecting member extending from said flat proximal end surface adapted to be mounted and fit within said central member dovetail mounting recess.

14. A sterile composite graft as claimed in claim 13 wherein at least one cap member is constructed of cortical bone.

15. A sterile composite graft as claimed in claim 13 wherein each cap member is ring shaped with a tapered cross section differing in height from front to rear said taper ranging from about 5 degrees to about 10 degrees.

16. A sterile composite graft as claimed in claim 13 wherein each cap member has a top surface which has a plurality of teeth formed thereon.

17. A sterile composite graft as claimed in claim 13 wherein said biocompatible plastic is PEEK.

18. A sterile composite grafts as claimed in 13 wherein at least one cap member is constructed of cortical bone.

19. A sterile composite graft as claimed in claim 13 wherein said ribs are V shaped.

20. A sterile composite graft as claimed in claim 13 wherein said central member defines a bore through its side wall transverse to a central axis of said central member.

21. A sterile composite bone graft for use in implants comprising:
a load bearing cylindrical center member having a ring shaped cross section constructed of biocompatible plastic and defining a cylindrical interior chamber, each end of said cylindrical center member defining a planar surface with a dovetail shaped recess formed in said planar surface, said ring shaped center member defining a plurality of stand alone ribs formed in a side wall, a plurality of cap members mounted to the ends of said center member, each of said cap member being constructed of allograft bone and formed with a tapered height, each said cap member defining a flat proximal bottom surface with a dovetail shaped member extending from said flat proximal end surface adapted to be mounted and fit within a central shaped member dovetail recess.

22. A sterile composite graft as claimed in claim 21 wherein said ribs form a V shape.

23. A sterile composite graft as claimed in claim 21 wherein each said cap member has a top surface which has a plurality of teeth formed thereon.

24. A sterile composite graft as claimed in claim 21 wherein said ribs are located adjacent said dovetail shaped recesses of said center member.

25. A sterile composite graft as claimed in claim 21 wherein said cap members and said center member define a through going channel running through each memeber.
